(19) United States
(12) Patent Application Publication
(10) Pub. No.: US 2022/0192945 A1
(43) Pub. Date: Jun. 23, 2022

(54) AQUEOUS LIQUID COSMETIC AND PEN TYPE COSMETIC PRODUCT

(71) Applicant: TOKIWA CORPORATION, Nakatsugawa-shi (JP)

(72) Inventor: Kazunori OGINO, Kawaguchi-shi (JP)

(21) Appl. No.: 17/548,592

(22) Filed: Dec. 13, 2021

(30) Foreign Application Priority Data
Dec. 23, 2020 (JP) 2020-213861

(51) Int. Cl. A61K 8/29; A61K 8/19; A61Q 1/10; A45D 34/04

(52) U.S. Cl. CPC A61K 8/29; A61K 8/19; A61Q 1/10; A45D 34/042; A61K 2800/5424; A61K 2800/412; A61K 2800/872; A61K 2800/95; A61K 2800/43

(57) ABSTRACT

An aqueous liquid cosmetic includes an inorganic coloring pigment, an anionic dispersant, and a film-forming polymer emulsion. The inorganic coloring pigment comprises a titanium black component having a primary particle size of 0.15 μm or more and at least one iron oxide component selected from the group consisting of red iron oxide, black iron oxide, and yellow iron oxide. Additionally, a content of the inorganic coloring pigment is 11 to 35% by mass based on a total amount of the cosmetic, and a content of the anionic dispersant is 8 parts by mass or less with respect to 100 parts by mass of a sum of the titanium black component and the at least one iron oxide component.

### CROSS-REFERENCE TO RELATED APPLICATIONS

This application claims the benefit of priority from Japanese Patent Application No. 2020-213861, filed on Dec. 23, 2020, the entire contents of which are incorporated herein by reference.

### TECHNICAL FIELD

The present invention relates to an aqueous liquid cosmetic and a pen type cosmetic product.

### BACKGROUND

As a product form of liquid cosmetics, autopen type cosmetic products from which a cosmetic is discharged by the action of surface tension of a liquid cosmetic and a capillary phenomenon have been utilized. For such cosmetic products, aqueous liquid cosmetics have been commonly used from the viewpoints that satisfactory discharge is likely to be obtained and that the drying rate after application is fast. On the other hand, aqueous liquid cosmetics tend to have inferior water resistance. In general, the water resistance of cosmetics such as eyeliners is enhanced using water-insoluble film-forming agents. In the case of aqueous liquid cosmetics, for example, in Japanese Unexamined Patent Publication No. 2003-073220, a liquid cosmetic obtained by blending an emulsion of a polymer and/or copolymer of a monomer selected from acrylic acid, an alkyl acrylate, methacrylic acid, an alkyl methacrylate, vinyl acetate, and styrene (except for polystyrene) has been suggested.

### SUMMARY

In recent years, demands for colors other than black and brown are increasing in liquid eyeliners, eyebrow cosmetics, and the like. Therefore, black pigments such as black iron oxide and carbon black may be mixed with inorganic pigments such as red iron oxide, yellow iron oxide, titanium oxide, and Prussian blue; and other pigments such as an organic colorant lake.

However, it has been found that when an aqueous liquid cosmetic containing the above-described mixed system of pigments is used as an autopen type product, shading difference or color difference may occur in the drawn lines.

The present invention was achieved in view of the above-described circumstances, and it is an object of the invention to provide an aqueous liquid cosmetic that can be discharged even when used as an autopen type product, also has sufficient water resistance, and can form drawn lines with suppressed shading difference and color difference, and to provide a pen type cosmetic product including the aqueous liquid cosmetic.

In order to solve the above-described problems, the present invention provides an aqueous liquid cosmetic including: (A) an inorganic coloring pigment; (B) an anionic dispersant; and (C) a coating film-forming polymer emulsion, wherein the content of the component (A) is 11% to 35% by mass based on the total amount of the cosmetic, the component (A) contains (A1) a titanium black having a primary particle size of 0.15 μm or more and (A2) at least one iron oxide selected from the group consisting of red iron oxide, black iron oxide, and yellow iron oxide, and the content of the component (B) is 8 parts by mass or less with respect to 100 parts by mass of the sum of the component (A1) and the component (A2).

According to the aqueous liquid cosmetic of the present invention, as the aqueous liquid cosmetic has the above-described configuration, the aqueous liquid cosmetic can be discharged even when used as an autopen type product, has sufficient water resistance, and can form drawn lines with suppressed shading difference and color difference. The inventors of the present invention speculate as follows about the reason why such effects are provided. With regard to an aqueous liquid cosmetic including the component (C), it is believed that when the component (A1) and the component (A2) are combined, and the component (B) is used at a specific proportion or less with respect to these components (A1) and (A2), the dispersed state of the component (A1) and the component (A2) can be maintained satisfactorily while suppressing a decrease in the water resistance attributable to the anionic component. It is speculated that as a result, sufficient discharge properties, water resistance of a makeup film, and the suppression of shading difference and color difference in the drawn lines can all be achieved in a well-balanced manner.

With regard to the above-described aqueous liquid cosmetic, from the viewpoint of discharge properties, the viscosity at 25° C. may be 50 mPa·s or less. In this case, the cosmetic is likely to be stably discharged by an autopen type application tool.

Furthermore, from the viewpoints of further enhancing color development, suppression of shading difference and color difference in drawn lines, and discharge properties, the mass ratio [(A1)/(A2)] between the component (A1) and the component (A2) may be 1/10 to 6/1.

The present invention provides a pen type cosmetic product including an autopen type application tool and the above-described aqueous liquid cosmetic filled in the application tool.

According to the present invention, an aqueous liquid cosmetic that can be discharged even when used as an autopen type product, has sufficient water resistance, and can form drawn line with suppressed shading difference and color difference, and a pen type cosmetic product including the aqueous liquid cosmetic, can be provided.

### BRIEF DESCRIPTION OF THE DRAWINGS

FIG. 1 is a schematic cross-sectional view for describing a pen type cosmetic product.

### DETAILED DESCRIPTION

The aqueous liquid cosmetic of the present embodiment includes: (A) an inorganic coloring pigment (hereinafter, may also be referred to as component (A)); (B) an anionic dispersant (hereinafter, may also be referred to as component (B)); and (C) a film-forming polymer emulsion (hereinafter, may also be referred to as component (C)).

Incidentally, the term aqueous according to the present specification means that at least water is included. An aqueous cosmetic may further include a lower alcohol having 1 to 5 carbon atoms, such as ethanol, in addition to water.

The content of water in the aqueous liquid cosmetic of the present embodiment may be 30% to 85% by mass, may be 40% to 80% by mass, or may be 50% to 70% by mass, based on the total amount of the cosmetic.

Furthermore, the term liquid according to the present specification implies that the cosmetic exhibits fluidity at 25° C. The viscosity at 25° C. of the aqueous liquid cosmetic of the present embodiment may be 10000 mPa·s or less and can be appropriately set according to the shape of the container. For example, regarding the pen type, a cosmetic having a viscosity at 25° C. of 5000 mPa·s or less is preferred.

From the viewpoint of making the discharge properties from an autopen type product satisfactory, the aqueous liquid cosmetic of the present embodiment is such that the viscosity at 25° C. may be 50 mPa·s or less, may be 40 mPa·s or less, may be 35 mPa·s or less, may be 30 mPa·s or less, or may be 25 mPa·s or less. Furthermore, from the viewpoint of usability, the aqueous liquid cosmetic of the present embodiment is such that the viscosity at 25° C. may be 4 mPa·s or more, or may be 6 mPa·s or more.

The above-described viscosity means a value measured for a sample at 25° C. using a Brookfield type viscometer (BM type) under the following conditions.

5 to 50 mPa·s: BL adaptor, speed of rotation 12 rpm

50 to 500 mPa·s: Rotor No. 1, speed of rotation 12 rpm

250 to 2500 mPa·s: Rotor No. 2, speed of rotation 12 rpm

1000 to 10000 mPa·s: Rotor No. 3, speed of rotation 12 rpm

5000 to 50000 mPa·s: Rotor No. 4, speed of rotation 12 rpm

[(A) Inorganic Coloring Pigment]

Regarding the inorganic coloring pigment, any inorganic coloring pigment that is usually used for cosmetic products can be used without particular limitation. Examples thereof include red iron oxide, yellow iron oxide, black iron oxide, cobalt oxide, chromium oxide, ultramarine blue, Prussian blue, titanium oxide, microparticulate titanium oxide, zinc oxide, titanium black (titanium-titanium oxide sintered product), carbon black, barium sulfate, and pearl pigments (titanated mica, iron oxide-coated titanium mica, microparticulate titanium oxide-coated titanated mica, barium sulfate-coated titanated mica, fish scale guanine, bismuth oxychloride, aluminum flakes, and the like). These powders may be subjected to a surface treatment for the purpose of enhancing usability, dispersibility, and the like. Examples of the surface treatment include a metal soap, a silicone compound, a fluorine compound, a surfactant, and an amino acid compound.

From the viewpoints of color development and suppression of shading difference and color difference in the drawn lines, the aqueous liquid cosmetic of the present embodiment can contain, as the component (A), (A1) a titanium black having a primary particle size of 0.15 μm or more (hereinafter, may be referred to as component (A1)) and (A2) at least one iron oxide selected from the group consisting of red iron oxide, black iron oxide, and yellow iron oxide (hereinafter, may be referred to as component (A2)).

Regarding the component (A1), for example, a commercially available product such as Tilack D (manufactured by AKO KASEI Co., LTD., trade name) can be used. The component (A1) may be a boron-free product.

From the viewpoints of color development and suppression of shading difference and color difference in the drawn lines, the primary particle size of the component (A1) may be 0.15 μm to 0.6 μm, may be 0.15 μm to 0.5 μm, or may be 0.2 μm to 0.4 μm.

From the viewpoints of color development and suppression of shading difference and color difference in the drawn lines, the content of the component (A1) may be 20% to 80% by mass, may be 22% to 70% by mass, may be 25% to 60% by mass, or may be 27% to 50% by mass, based on the total amount of the component (A).

From the viewpoints of color development and suppression of shading difference and color difference in the drawn lines, the content of the component (A2) may be 20% to 70% by mass, may be 25% to 65% by mass, or may be 30% to 60% by mass, based on the total amount of the component (A).

From the viewpoint of achieving all of water resistance, color development, suppression of shading difference and color difference in the drawn lines, discharge properties, and dispersion stability at high levels in a well-balanced manner, the total content of the component (A1) and the component (A2) may be 15% to 30% by mass, may be 16% to 25% by mass, or may be 17% to 21% by mass, based on the total amount of the cosmetic.

From the viewpoint of further enhancing color development, suppression of shading difference and color difference in the drawn lines, and discharge properties, the mass ratio [(A1)/(A2)] between the component (A1) and the component (A2) may be 1/10 to 6/1 or may be 1/3 to 6/1.

From the viewpoint of enhancing discharge properties, the mass ratio [(A1)/(A2)] between the component (A1) and the component (A2) may be 1/1 to 6/1, may be 1/1 to 3/1, or may be 1/1 to 2/1.

From the viewpoints of color development, suppression of shading difference and color difference in the drawn lines, and dispersion stability, the mass ratio [(A1)/(A2)] between the component (A1) and the component (A2) may be 1/10 to 1/1, may be 1/5 to 1/1, may be 1/3 to 1/1, or may be 2/5 to 1/1.

The aqueous liquid cosmetic of the present embodiment may further contain (A3) titanium oxide (hereinafter, may also be referred to as component (A3)) as the component (A), in addition to the above-mentioned component (A1) and component (A2).

From the viewpoint of expressing various colors of the drawn lines and imparting concealability, the content of the component (A3) may be 0.01% to 30% by mass, may be 1% to 28% by mass, or may be 5% to 27% by mass, based on the total amount of the component (A).

Furthermore, from the viewpoint of expression various colors of the drawn lines and imparting concealability, the content of the component (A3) may be 0 to 60 parts by mass, may be 3 to 50 parts by mass, or may be 5 to 40 parts by mass, with respect to 100 parts by mass of the sum of the component (A1) and the component (A2).

From the viewpoint of forming drawn lines with suppressed shading difference and color difference, in the aqueous liquid cosmetic of the present embodiment, the content of carbon black may be 10 parts by mass or less with respect to 100 parts by mass of the component (A), or the aqueous liquid cosmetic may not include carbon black.

The content of the component (A) in the aqueous liquid cosmetic of the present embodiment may be 11% to 35% by mass, may be 15% to 30% by mass, or may be 18% to 28% by mass, based on the total amount of the cosmetic. When the content of the component (A) is in such a range, drawn lines with suppressed shading difference and color difference can be formed, and a cosmetic that is excellent in terms of discharge properties and color development is likely to be obtained.

[(B) Anionic Dispersant]

Examples of the component (B) include anionic polymers such as polycarboxylates such as a polyaspartate, a polyacryate, and a polymethacrylate. Furthermore, regarding the component (B), a water-soluble compound can be used.

A polyaspartate is a polymer including aspartic acid as a constituent unit. A polycarboxylate is a homopolymer or copolymer including a constituent unit derived from an ethylenically unsaturated carboxylic acid such as acrylic acid, methacrylic acid, maleic acid, or itaconic acid. The polyacrylate is a homopolymer including acrylic acid as a constituent unit, and a polymethacrylate is a homopolymer including methacrylic acid as a constituent unit.

Regarding a salt of the polymer, an alkali metal salt such as a sodium salt or a potassium salt; an organic amine salt such as a diethanolamine salt or a triethanolamine salt; or a basic amino acid salt can be utilized. The salt of the polymer may be a sodium salt or a potassium salt.

Regarding the component (B), one kind thereof can be used singly, or two or more kinds thereof can be used in combination.

The average molecular weight (Mw) of the anionic polymer may be 1000 to 15000 or may be 2000 to 10000. The average molecular weight (Mw) of the water-soluble polymer can be measured using gel permeation chromatography (GPC) by employing polyethylene glycols having known molecular weights as reference materials.

For the anionic dispersant, a commercially available product can be used. Examples of the polyaspartate include AQUADEW SPA-30 (manufactured by Ajinomoto Co., Inc., average molecular weight (Mw) 4000) and Baypure DS100 (manufactured by LANXESS AG, average molecular weight (Mw) 2500). Examples of the polyacrylate include ARON T-50 (manufactured by TOAGOSEI CO., LTD., average molecular weight (Mw) 6000) and ARON A-30SL (manufactured by TOAGOSEI CO., LTD., average molecular weight (Mw) 6000). Examples of the polycarboxylate include ARON A-6330 (manufactured by TOAGOSEI CO., LTD., average molecular weight (Mw) 10000) and ARON A-6001 (manufactured by TOAGOSEI CO., LTD., average molecular weight (Mw) 8000).

From the viewpoints of having discharge properties and sufficient water resistance and forming drawn lines with suppressed shading difference and color difference, the content of the component (B) may be 8 parts by mass or less, may be 0.1 to 6 parts by mass, may be 1 to 5 parts by mass, or may be 1.5 to 3 parts by mass, with respect to 100 parts by mass of the component (A). Furthermore, from similar viewpoints, the content of the component (B) may be 8 parts by mass or less, 0.5 to 5 parts by mass, 1 to 3 parts by mass, or 1.5 to 3 parts by mass, with respect to 100 parts by mass of the sum of the component (A1) and the component (A2).

From the viewpoint of water resistance, the content of the component (B) may be 3 to 35 parts by mass, may be 4 to 30 parts by mass, or 5 to 10 parts by mass, with respect to 100 parts by mass of the content of the component (C) in terms of the solid content concentration.

The content of the component (B) in the aqueous liquid cosmetic of the present embodiment may be 0.1% to 1.5% by mass, may be 0.2% to 1% by mass, or may be 0.3% to 0.8% by mass, based on the total amount of the cosmetic.

[(C) Film-Forming Polymer Emulsion]

The polymer included in the film-forming polymer emulsion may be a water-insoluble polymer or copolymer including an alkyl (meth)acrylate monomer as a constituent unit. Examples of the constituent unit of the copolymer include a vinyl acetate monomer and a styrene monomer. In the case of a copolymer, the copolymer may be a random copolymer, a graft copolymer, a block copolymer, or a core-shell type copolymer.

Specific examples of the film-forming polymer emulsion include an alkyl acrylate copolymer emulsion, an alkyl acrylate-styrene copolymer emulsion, and an alkyl acrylate-vinyl acetate copolymer emulsion. The alkyl acrylate as used herein also includes an alkyl methacrylate. Regarding the film-forming polymer emulsion, a polymer emulsion including water as a medium and having a solid content concentration of 30% to 60% by mass can be used.

Regarding the film-forming polymer emulsion, a commercially available product can be used. Examples of the alkyl acrylate copolymer emulsion include YODOSOL GH800F (manufactured by Akzo Nobel N.V., product name, solid content concentration 45% by mass), YODOSOL GH810F (manufactured by Akzo Nobel N.V., product name, solid content concentration 46% by mass), YODOSOL GH34F (manufactured by Akzo Nobel N.V., product name, solid content concentration 42% by mass), and DAITOSOL 5000SJ (manufactured by DAITO KASEI KOGYO CO., LTD., product name, solid content concentration 50% by mass). Examples of the alkyl acrylate-styrene copolymer emulsion include YODOSOL GH41F (manufactured by Akzo Nobel N.V., product name, solid content concentration 45% by mass), DAITOSOL 5000STY (manufactured by DAITO KASEI KOGYO CO., LTD., product name, solid content concentration 50% by mass), and EMUPOLY CE-119N (sold by Nikko Chemicals Co., Ltd., product name). Examples of the alkyl acrylate-vinyl acetate copolymer emulsion include VINYSOL 2140L (manufactured by Daido Chemical Corporation, product name, solid content concentration 45% by mass).

The film-forming polymer emulsion can be used singly, or two or more kinds thereof can be used in combination.

The content of the component (C) in the aqueous liquid cosmetic of the present embodiment may be, in terms of the solid content concentration, 3% to 18% by mass, 5% to 15% by mass, may be 6% to 12% by mass, or may be 7% to 10% by mass, based on the total amount of the cosmetic. When the content of the component (C) is in the above-described range, securement of discharge properties and suppression of shading difference and color difference in the drawn lines are facilitated while enhancing the water resistance of a makeup film of the drawn lines and the like.

In the aqueous liquid cosmetic according to the present embodiment, other components that are usually used in cosmetic products in addition to each of the above-described components, for example, a powder other than the component (A), a surfactant, a water-soluble dispersant other than the component (B), a film-forming agent other than the component (C), a humectant, a viscosity adjusting agent, an antiseptic agent, a pH adjusting agent, a chelating agent, an ultraviolet absorber, vitamins, a beauty component, an antioxidant, and a fragrance can be appropriately blended in as necessary, to the extent that does not impair the effects of the present invention.

Examples of the powder other than the component (A) include organic pigment powders such as Red No. 201, Red No. 202, Red No. 205, Red No. 226, Red No. 228, Orange No. 203, Orange No. 204, Blue No. 404, and Yellow No. 401; Organic pigment powders such as zirconium, barium, or aluminum lakes, such as Red No. 3, Red No. 104,

Red No. 106, Orange No. 205, Yellow No. 4, Yellow No. 5, Green No. 3, and Blue No. 1; white extender powders such as carmine, talc, muscovite, phlogopite, lepidolite, biotite, synthetic mica, sericite, synthetic sericite, kaolin, silicon carbide, bentonite, smectite, diatomaceous earth, aluminum silicate, aluminum magnesium metasilicate, calcium silicate, barium silicate, magnesium silicate, calcium carbonate, magnesium carbonate, hydroxyapatite, and boron nitride; synthetic resin powders such as a polyamide-based resin, a polyethylene-based resin, a polyacrylic resin, a polyester-based resin, a fluorine-based resin, a cellulose-based resin, a polystyrene-based resin, and a styrene-acrylic copolymerized resin; organic polymer resin powders such as a polypropylene-based resin and a urethane resin; organic low molecular compound powders such as zinc stearate and N-acyl lysine; and natural organic powders such as a silk powder and a cellulose powder.

Regarding the surfactant, a hydrophilic nonionic surfactant, an anionic surfactant, a cationic surfactant, an amphoteric surfactant, and the like can be used.

Examples of the hydrophilic nonionic surfactant include a polyoxyalkylene alkyl ether, a glycerin alkyl ether, a glycerin fatty acid ester, a polyglycerin fatty acid ester, a sorbitan fatty acid ester and an alkylene glycol adduct thereof, a polyalkylene glycol fatty acid ester, a polyglycerin-modified silicone, and a polyether-modified silicone. Examples of the anionic surfactant include an alkyl phosphate, a polyoxyalkylene alkyl ether phosphate, a sulfonate, and an alkyl sulfate. Examples of the cationic surfactant include an alkylamine salt and an alkyltrimethylammonium salt. Examples of the amphoteric surfactant include lecithin, a carbobetaine type amphoteric surfactant, a sulfobetaine type amphoteric surfactant, and an amino acid type amphoteric surfactant.

Regarding a water-soluble dispersant other than the component (B), a water-soluble polymer such as polyvinylpyrrolidone (PVP), polyvinyl alcohol (PVA), or a vinylpyrrolidone-vinyl acetate copolymer (VP-VA copolymer) can be used.

Examples of the film-forming agent other than the component (C) include PVP, PVA, and a VP-VA copolymer.

Examples of the viscosity adjusting agent include clay minerals and polysaccharides such as xanthan gum.

The aqueous liquid cosmetic according to the present embodiment can be produced by dissolving or dispersing the above-mentioned component (A), component (B), component (C), water, and other components as necessary, and uniformly stirring and mixing the components.

The aqueous liquid cosmetic according to the present embodiment can be used as a makeup cosmetic for eyeliner, eyebrow pencil, eyeshadow, mascara, or the like.

The aqueous liquid cosmetic according to the present embodiment can be utilized as a known product form that is used in cosmetic products. Examples of the product form include a pen type and a bottle type. Examples of the pen type include a mechanical type which includes a cosmetic storage part such as a fiber bundle impregnated with a cosmetic, or a filling part filled with a cosmetic, and an application part joined with this cosmetic storage part, the application part being composed of a brush or a felt, and in which the cosmetic is forcibly discharged by applying force to the storage part by dialing, knocking, or the like; and an autopen type in which a cosmetic is discharged by the action of the surface tension of a liquid cosmetic and a capillary phenomenon.

From the viewpoints of convenience of the method of use and portability, it is preferable that the aqueous liquid cosmetic according to the present embodiment is utilized as an autopen type product. Regarding the container for the autopen type product, any known container can be used, and for example, the liquid cosmetic container disclosed in Japanese Unexamined Patent Publication No. 2016-87094 may be mentioned.

The pen type cosmetic product of the present embodiment includes an autopen type application tool and the above-mentioned aqueous liquid cosmetic according to the present embodiment filled in the application tool.

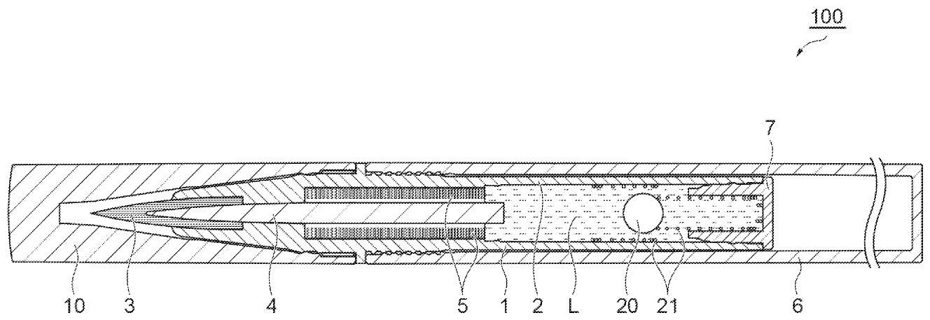

FIG. 1 is a schematic cross-sectional view showing a pen type cosmetic product. The pen type cosmetic product 100 shown in FIG. 1 is such that the overall shape represents a slender round bar shape similar to a wiring tool, and the pen type cosmetic product 100 roughly includes a cylindrical-shaped container main body 1; a storage part 2 provided inside the container main body 1 and storing the aqueous liquid cosmetic L according to the present embodiment; a brush 3 placed at the front end of the container main body 1 for applying the aqueous liquid cosmetic L in the storage part 2; a shaft-shaped relay core 4 disposed inside the container main body 1 and connecting the storage part 2 and the brush 3; and an approximately cylindrical-shaped bellows-like member 5 placed around the relay core 4. Here, in order to allow the user to apply the cosmetic by holding the container main body 1, a bottomed cylinder-shaped gripping cylinder 6 is attachably and detachably fitted to the container main body 1 by means of screws. The container main body 1 is not limited to a cylindrical shape and may be a rectangular tube or the like.

With regard to the pen type cosmetic product 100, the storage part 2, the brush 3, and the relay core 4 constitute an autopen type application tool, and the aqueous liquid cosmetic according to the present embodiment is filled in the storage part 2.

The container main body 1 is formed from, for example, PP or the like and configured into a tapered cylindrical shape with brim. The front-end face of the gripping cylinder 6 screwed to the container main body 1 is butted against the rear-end face of the brim part provided on the outer circumferential surface of the container main body 1, and an opened end face of a cap 10 to be mounted on the container main body 1 is butted against the front-end face of the brim part. Furthermore, the opening at the rear end of the container main body 1 is closed by placing a bottomed cylindrical-shaped tail plug 7 to be thrusted in.

The bellows-like member 5 is intended for controlling the flow rate of the aqueous liquid cosmetic L, and this bellows-like member 5 has grooves (bellows) including the aqueous liquid cosmetic L and is mounted on the container main body 1 as the cylindrical-shaped rear end part of the member is fitted into a recess part on the inner circumferential surface of the container main body 1. Furthermore, the storage part 2 is formed between the rear end part of the bellows-like member 5 and the tail plug 7 in the container main body 1, and the aqueous liquid cosmetic L is filled in this storage part 2.

The relay core 4 is formed from, for example, an acrylic resin or the like, and the relay core 4 extends in the axial direction so as to pass through a tubular hole of the bellows-like member 5 and is mounted on the bellows-like member 5 as the front-end side of the relay core 4 is fitted into the front-end side of the bellows-like member 5. The relay core 4 connects the inside of the storage part 2 with the brush 3, as the rear-end side portion of the relay core 4 enters the inside of the storage part 2 while the front-end side portion thereof enters the brush 3. Then, the relay core 4 enables suctioning of the aqueous liquid cosmetic L inside the storage part 2 and supplying the aqueous liquid cosmetic L to the brush 3 by means of the capillary phenomenon.

With regard to the pen type cosmetic product 100 show in FIG. 1, the applicator is formed of a brush; however, the applicator may be changed to a felt tip or a urethane tip.

At the front-end side of the container main body 1, a bottomed cylindrical-shaped cap 10 for protecting the brush 3 or and the like is attachably and detachably mounted by interdigitation.

In the pen type cosmetic product 100, a stirring bar 20 that can move in the axial direction and a coil spring 21 that can stretch in the axial direction are housed, together with the aqueous liquid cosmetic L, in the storage part 2. The stirring bar 20 as used herein is formed into a spherical shape; however, a polyhedral, a pyramid, or the like may also be adopted.

The coil spring 21 is an integrally molded spring in which a plurality of spring parts having diameter differences (here, two spring parts having a diameter difference) are integrally linked in the axial direction, and in this case, for example, the spring is molded from SUS or the like. This coil spring 21 includes a small diameter spring part having a smaller diameter than the diameter of the stirring bar 20 in the latter half and also includes a large diameter spring part having a larger diameter than the diameter of the stirring bar 20 near the axial direction in front of the small diameter spring part.

Here, when a user shakes the pen type cosmetic product 100, since the stirring bar 20 is capable of moving in the axial direction inside the large diameter spring part that constitutes the coil spring 21, the stirring bar 20 moves in the axial direction, and the aqueous liquid cosmetic L is stirred due to the movement of this stirring bar 20.

In the pen type cosmetic product 100 configured as such, the aqueous liquid cosmetic L in the storage part 2 flows out toward the brush 3 on the front-end side via the relay core 4 and is provided for application by the user by means of the brush 3. Incidentally, since the pen type cosmetic product 100 according to the present embodiment includes the stirring bar 20 and the coil spring 21 mentioned above, the aqueous liquid cosmetic L can be made to efficiently flow out to the brush 3; however, the pen type cosmetic product 100 may also be changed to a configuration that does not include a stirring bar 20 and a coil spring 21.

Thus, a cosmetic product including a container having a so-called direct liquid type configuration has been described as an example of the pen type cosmetic product according to the present embodiment; however, the container of the cosmetic product is not particularly limited to one having such a structure, and any known container can be used. For example, a container having a so-called inner cotton type configuration, in which a bellows-like member 5, a stirring bar 20, and a coil spring 21 of the pen type cosmetic product 100 are omitted, a storage part 2 houses an inner cotton member filled with the aqueous liquid cosmetic L by impregnation, and the aqueous liquid cosmetic L is suctioned and supplied to a brush 3 by a relay core 4 whose rear-end side portion is made to enter the inner part of the inner cotton member, may also be used.

### EXAMPLES

Hereinafter, the present invention will be described more specifically by way of Examples; however, the present invention is not intended to be limited to the following Examples.

<Production of Aqueous Liquid Cosmetic>

### Examples 1 to 9 and Comparative Examples 1 to 7

The various components shown in Tables 1 to 4 were mixed with a Disper at the proportions (% by mass) indicated in the same tables, and each aqueous liquid cosmetic was obtained. In the tables, the values for the polymer emulsion, the anionic dispersant, and the nonionic dispersant represent the blending amounts of solid contents.

Regarding the various components shown in Tables 1 to 4, materials shown below were used.

Titanium black-1: Titanium black (manufactured by AKO KASEI Co., LTD., product name “Tilack D TM-B-CSM”, primary particle size: 0.2 to 0.4 μm)

Titanium black-2: Titanium black (manufactured by AKO KASEI Co., LTD., product name “Tilack D TS-A-CSM”, primary particle size: 0.03 to 0.1 μm)

Na polyaspartate: Na polyaspartate (manufactured by Ajinomoto Co., Inc., product name “AQUADEW SPA-30B”, average molecular weight (Mw) 4000)

Na polyacrylate: Na polyacrylate (manufactured by TOAGOSEI CO., LTD., product name “ARON T-50”, average molecular weight (Mw) 6000) Na polycarboxylate: Na polyacrylate (manufactured by

TOAGOSEI CO., LTD., product name “ARON A-6330”, average molecular weight (Mw) 10000)

Polymer emulsion 1: Alkyl acrylate copolymer emulsion (manufactured by Akzo Nobel N.V., product name “YODOSOL GH800F”, solid content concentration 45% by mass)

Nonionic dispersant-1: Beheneth-30 (polyoxyethylene behenyl ether, manufactured by Nikko Chemicals Co., Ltd., product name “NIKKOL BB-30”)

<Viscosity of Aqueous Liquid Cosmetic>

For a sample at 25° C., viscosity was measured using a Brookfield type viscometer (BM type) under the following conditions. The measuring time was set to 1 minute.

5 to 50 mPa·s: BL adaptor, speed of rotation 12 rpm

50 to 500 mPa·s: Rotor No. 1, speed of rotation 12 rpm

250 to 2500 mPa·s: Rotor No. 2, speed of rotation 12 rpm

1000 to 10000 mPa·s: Rotor No. 3, speed of rotation 12 rpm

5000 to 50000 mPa·s: Rotor No. 4, speed of rotation 12 rpm

<Evaluation of Aqueous Liquid Cosmetic>

For the aqueous liquid cosmetics obtained as described above, discharge properties, shading difference, water resistance, dispersion stability, and color development were evaluated by the following evaluation methods.

(Discharge Properties)

An aqueous liquid cosmetic obtained as described above was filled in a storage part of an autopen type liquid eyeliner container (applicator: brush, relay core: acrylic resin) having a structure similar to that of the container shown in FIG. 1 to product a filled product.

A container filled product was used to write on the back of the hand, the satisfactoriness of discharge at the time of applying three lines each measuring 2 mm in width and 4 cm in length was visually inspected, the discharge properties were evaluated according to the following evaluation criteria.

[Evaluation Criteria]

A: Discharge is achieved with a sufficient liquid amount, and the drawn lines are uniform.

B: Discharge is achieved with an acceptable liquid amount, and the drawn lines are almost uniform.

C: Because the liquid amount discharged is small, the drawn lines are slightly uneven.

D: The liquid amount discharged is small, and the drawn lines are scratchy.

(Shading Difference)

Ten expertise panels for cosmetics evaluation were asked to use the container filled products obtained as described above, and the shading difference of drawn lines was evaluated. Regarding the evaluation of the shading difference, a 5-grade evaluation was carried out according to the following evaluation criteria, rating scores were assigned for each sample, and the average score of the rating scores obtained from all the panels was determined according to the following determination criteria.

[Rating Score: Evaluation Criteria]

Score 5: There is no shading difference at all in the drawn lines.

Score 4: There is almost no shading difference in the drawn lines.

Score 3: There is slight shading difference in the draw lines.

Score 2: There is shading difference in the drawn lines.

Score 1: There is shading difference to a considerable extent in the drawn lines.

[Determination Criteria (Average Score of Rating Scores)]

A: 4 or greater

B: 3 or greater and less than 4

C: 2 or greater and less than 3

D: Less than 2

(Water Resistance)

The cosmetic was applied on the arm and dried for 10 minutes, subsequently the cosmetic was wetted with flowing water and rubbed with the ball of a finger, the condition of residue of the applied material was visually observed, and water resistance was evaluated according to the following evaluation criteria.

[Evaluation Criteria]

A: There is no peeling at the applied portion (highly satisfactory).

B: There is little peeling at the applied portion (satisfactory).

C: Slight peeling occurred at the applied portion (slightly poor).

D: Significant peeling occurred at the applied portion (poor).

(Dispersion Stability (Unlikeliness of Sedimentation))

A sample was introduced into a transparent bottle and was left to stand for one day in an environment at 25° C., the state of sedimentation of powder components was visually inspected, and the dispersion stability (unlikeliness of sedimentation) was evaluated according to the following evaluation criteria.

[Evaluation Criteria]

A: Powder can be visually recognized in the entire portion of the liquid.

B: Powder can be visually recognized up to a middle portion of the liquid.

C: Powder can be visually recognized in the lower portion of the liquid.

D: Powder can be visually recognized only at the bottom portion of the liquid.

(Color Development)

A container filled product obtained as described above was used to write on the back of the hand, color development (concealability) of drawn lines was visually inspected, and the color development was evaluated according to the following evaluation criteria.

[Evaluation Criteria]

A: The drawn lines sufficiently develop color and conceal the skin.

B: The drawn lines develop color and almost conceal the skin.

C: Color development of the drawn lines is not sufficient, and skin concealment is slightly weak.

D: Color development of the drawn lines is weak, and the drawn lines do not conceal the skin.

TABLE 1

| | | | Example | | | | |
|---|---|---|---|---|---|---|---|
| | | | 1 | 2 | 3 | 4 | 5 |
| Component (A) | Component (A1) | Titanium black-1 | 7 | 11 | 4 | 15 | 7 |
| | Titanium black-2 | | — | — | — | — | — |
| | Component (A2) | Black iron oxide | 4 | 6 | 2 | — | 4 |
| | | Red iron oxide | 4 | 8 | 3 | 10 | 4 |
| | | Yellow iron oxide | 2 | 4 | 1 | — | 2 |
| | Component (A3) | Titanium oxide | 6 | 6 | 1 | 2 | 6 |

| Component (B) | Na polyaspartate | 0.5 | 0.5 | 0.5 | 0.5 | — |
|---|---|---|---|---|---|---|
| | Na polyacrylate | — | — | — | — | 0.5 |
| | Na polycarboxylate | — | — | — | — | — |
| Component (C) | Polymer emulsion-1 | 8 | 8 | 8 | 8 | 8 |
| Others | Xanthan gum | 0.02 | 0.02 | 0.02 | 0.02 | 0.02 |
| | Na diethylhexyl sulfosuccinate | 0.3 | 0.3 | 0.3 | 0.3 | 0.3 |
| | Nonionic dispersant-1 | 0.5 | 0.5 | 0.5 | 0.5 | 0.5 |
| | Citric acid | 0.04 | 0.04 | 0.04 | 0.04 | 0.04 |
| | 1,3-Butylene glycol | 4 | 4 | 4 | 4 | 4 |
| | Phenoxyethanol | 0.3 | 0.3 | 0.3 | 0.3 | 0.3 |
| | Anhydrous ethanol | 3 | 3 | 3 | 3 | 3 |
| | Purified water | Balance | Balance | Balance | Balance | Balance |
| Content of component (A) (% by mass) | | 23.00 | 35.00 | 11.00 | 27.00 | 23.00 |
| Component (A1)/component (A2) (mass ratio) | | 0.7 | 0.6 | 0.7 | 1.5 | 0.7 |
| Component (B)/[component (A1) + component (A2)] (mass ratio) | | 0.03 | 0.02 | 0.05 | 0.02 | 0.03 |
| Viscosity @25° C. (mPa · s) | | 15 | 23 | 8 | 16 | 14 |
| Evaluation | Discharge properties | A | B | A | A | A |
| | Shading difference | A | A | A | B | A |
| | Water resistance | A | A | B | A | A |
| | Dispersion stability | A | A | A | B | A |
| | Color development | A | A | B | B | A |

TABLE 2

| | | | Example | | | |
|---|---|---|---|---|---|---|
| | | | 6 | 7 | 8 | 9 |
| Component (A) | Component (A1) | Titanium black-1 | 7 | 9 | 7 | 4 |
| | Titanium black-2 | | — | — | — | — |
| | Component (A2) | Black iron oxide | 4 | 4.5 | 4 | 2 |
| | | Red iron oxide | 4 | 4.5 | 4 | 3 |
| | | Yellow iron oxide | 2 | 2.5 | 2 | 1 |
| | Component (A3) | Titanium oxide | 6 | — | 6 | 1 |

| Component (B) | Na polyaspartate | — | 0.5 | 0.2 | 0.8 |
|---|---|---|---|---|---|
| | Na polyacrylate | — | — | — | — |
| | Na polycarboxylate | 0.5 | — | — | — |
| Component (C) | Polymer emulsion-1 | 8 | 8 | 5 | 12 |
| Others | Xanthan gum | 0.02 | 0.02 | 0.02 | 0.01 |
| | Na diethylhexyl sulfosuccinate | 0.3 | 0.3 | 0.3 | 0.5 |
| | Nonionic dispersant-1 | 0.5 | 0.5 | 0.5 | 0.5 |
| | Citric acid | 0.04 | 0.04 | 0.04 | 0.04 |
| | 1,3-Butylene glycol | 4 | 4 | 4 | 4 |
| | Phenoxyethanol | 0.3 | 0.3 | 0.3 | 0.3 |
| | Anhydrous ethanol | 3 | 3 | 3 | 3 |
| | Purified water | Balance | Balance | Balance | Balance |
| Content of component (A) (% by mass) | | 23.00 | 20.50 | 23.00 | 11.00 |
| Component (A1)/component (A2) (mass ratio) | | 0.7 | 0.8 | 0.7 | 0.7 |
| Component (B)/[component (A1) + component (A2)] (mass ratio) | | 0.03 | 0.02 | 0.01 | 0.08 |
| Viscosity @25° C. (mPa · s) | | 15 | 15 | 12 | 18 |
| Evaluation | Discharge properties | A | A | A | A |
| | Shading difference | A | A | B | A |
| | Water resistance | A | A | A | B |
| | Dispersion stability | A | A | B | A |
| | Color development | A | A | A | B |

TABLE 3

| | | | Comparative Example | | | | |
|---|---|---|---|---|---|---|---|
| | | | 1 | 2 | 3 | 4 | 5 |
| Component (A) | Component (A1) | Titanium black-1 | — | 17 | 3.5 | 16 | 7 |
| | Titanium black-2 | | — | — | — | — | — |
| | Component (A2) | Black iron oxide | 11 | — | 2 | 6 | 4 |
| | | Red iron oxide | 4 | — | 2 | 8 | 4 |
| | | Yellow iron oxide | 2 | — | 1 | 4 | 2 |
| | Component (A3) | Titanium oxide | 6 | 6 | 2 | 6 | 6 |

| Component (B) | Na polyaspartate | 0.5 | 0.5 | 0.5 | 0.5 | — |
|---|---|---|---|---|---|---|
| | Na polyacrylate | — | — | — | — | — |
| | Na polycarboxylate | — | — | — | — | — |
| Component (C) | Polymer emulsion-1 | 8 | 8 | 8 | 8 | 8 |
| Others | Xanthan gum | 0.02 | 0.02 | 0.02 | 0.02 | 0.02 |
| | Na diethylhexyl sulfosuccinate | 0.3 | 0.3 | 0.3 | 0.3 | 0.3 |
| | Nonionic dispersant-1 | 0.5 | 0.5 | 0.5 | 0.5 | 0.5 |
| | Citric acid | 0.04 | 0.04 | 0.04 | 0.04 | 0.04 |
| | 1,3-Butylene glycol | 4 | 4 | 4 | 4 | 4 |
| | Phenoxyethanol | 0.3 | 0.3 | 0.3 | 0.3 | 0.3 |
| | Anhydrous ethanol | 3 | 3 | 3 | 3 | 3 |
| | Purified water | Balance | Balance | Balance | Balance | Balance |
| Content of component (A) (% by mass) | | 23.00 | 23.00 | 10.50 | 40.00 | 23.00 |
| Component (A1)/component (A2) (mass ratio) | | 0 | — | 0.7 | 0.9 | 0.7 |
| Component (B)/[component (A1) + component (A2)] (mass ratio) | | 0.03 | 0.03 | 0.06 | 0.01 | 0.00 |
| Viscosity @25° C. (mPa · s) | | 15 | 16 | 6 | 30 | 35 |
| Evaluation | Discharge properties | A | A | A | D | D |
| | Shading difference | D | C | D | B | D |
| | Water resistance | A | A | B | A | A |
| | Dispersion stability | D | C | D | B | D |
| | Color development | A | D | D | A | A |

TABLE 4

| | | | Comparative Example | |
|---|---|---|---|---|
| | | | 6 | 7 |
| Component | Component | Titanium | | 7 |
| (A) | (A1) | black-1 | | |
| | Titanium black-2 | | 7 | |
| | Component | Black iron | 4 | 4 |
| | (A2) | oxide | | |
| | | Red iron | 4 | 4 |
| | | oxide | | |
| | | Yellow iron | 2 | 2 |
| | | oxide | | |
| | Component | Titanium | 6 | 6 |
| | (A3) | oxide | | |

| | | | |
|---|---|---|---|
| Component | Na polyaspartate | 0.5 | 2 |
| (B) | Na polyacrylate | | |
| | Na polycarboxylate | | |
| Component | Polymer emulsion-1 | 8 | 8 |
| (C) | | | |
| Others | Xanthan gum | 0.02 | 0.02 |
| | Na diethylhexyl | 0.3 | 0.3 |
| | sulfosuccinate | | |
| | Nonionic dispersant-1 | 0.5 | 0.5 |
| | Citric acid | 0.04 | 0.04 |
| | 1,3-Butylene glycol | 4 | 4 |
| | Phenoxyethanol | 0.3 | 0.3 |
| | Anhydrous ethanol | 3 | 3 |
| | Purified water | Balance | Balance |

| Content of component (A) | | 23.00 | 23.00 |
|---|---|---|---|
| (% by mass) | | | |
| Component (A1)/component (A2) | | 0 | 0.7 |
| (mass ratio) | | | |
| Component (B)/[component (A1) + | | 0.03 | 0.12 |
| component (A2)] mass ratio) | | | |
| Viscosity @ 25° C. (mPa · s) | | 12 | 7 |
| Evaluation | Discharge properties | A | A |
| | Shading difference | D | B |
| | Water resistance | A | D |
| | Dispersion stability | A | A |
| | Color development | D | A |

### REFERENCE SIGNS LIST

1: container main body, 2: storage part, 3: brush (applicator), 20: stirring bar, 21: coil spring (small diameter spring part and large diameter spring part), 100: pen type cosmetic product, L: aqueous liquid cosmetic.

What is claimed is:

1. An aqueous liquid cosmetic comprising:
an inorganic coloring pigment;
an anionic dispersant; and
a film-forming polymer emulsion,
wherein the inorganic coloring pigment comprises a titanium black component having a primary particle size of 0.15 μm or more and at least one iron oxide component selected from the group consisting of red iron oxide, black iron oxide, and yellow iron oxide,
wherein a content of the inorganic coloring pigment is 11 to 35% by mass based on a total amount of the cosmetic, and
wherein a content of the anionic dispersant is 8 parts by mass or less with respect to 100 parts by mass of a sum of the titanium black component and the at least one iron oxide component.

2. The aqueous liquid cosmetic according to claim 1, having a viscosity of 50 mPa·s or less at 25° C.

3. The aqueous liquid cosmetic according to claim 1, wherein a mass ratio of the titanium black component to the at least one iron oxide component is 1/10 to 6/1.

4. The aqueous liquid cosmetic according to claim 1, wherein the anionic dispersant comprises an anionic polymer having an average molecular weight (Mw) of 1000 to 15000.

5. The aqueous liquid cosmetic according to claim 1, wherein a content of the film-forming polymer emulsion is 3% to 18% by mass based on a total solid content of the cosmetic.

6. The aqueous liquid cosmetic according to claim 1, wherein the cosmetic does not comprise carbon black.

7. A pen-shaped cosmetic product comprising an automatic pen shaped applicator and the aqueous liquid cosmetic according to claim 1 filled in the applicator.
